(19) United States
(12) Patent Application Publication
(10) Pub. No.: US 2022/0383189 A1
(43) Pub. Date: Dec. 1, 2022

(54) METHODS AND SYSTEMS FOR PREDICTING COGNITIVE LOAD

(71) Applicant: Apple Inc., Cupertino, CA (US)

(72) Inventors: Joseph Yitan Cheng, Santa Clara, CA (US); Amruta Pai, Houston, TX (US); Erdrin Azemi, San Mateo, CA (US); Matthias R. Hohmann, Tubingen (DE)

(73) Assignee: Apple Inc., Cupertino, CA (US)

(21) Appl. No.: 17/554,895

(22) Filed: Dec. 17, 2021

Related U.S. Application Data
(60) Provisional application No. 63/194,407, filed on May 28, 2021.

(51) Int. Cl. G06N 20/00; G06N 5/02

(52) U.S. Cl. CPC G06N 20/00; G06N 5/02

(57) ABSTRACT

Methods and systems are provided for predicting cognitive load. A computing device receives sensor measurements from sensors. The sensor measurements correspond to characteristics of a user during the performance of a task. For each sensor, the computing device derives, from the sensor measurements of the sensor, a set of features predictive of the cognitive load of the user; generates, from those features, a self-attention vector that characterizes each feature of the set of features relative to another feature; and defines a feature vector from the features and the self-attention vector. The computing device generates an input feature vector from the feature vector of at least one sensor. The computing device then uses a machine-learning model to generate an indication of the cognitive load of the user during the performance of a task from the feature vector.

### CROSS-REFERENCE TO RELATED APPLICATIONS

This application claims the benefit of and the priority to U.S. Provisional Application No. 63/194,407, filed on May 28, 2021 and entitled, “Methods and Systems for Predicting Cognitive Load, which is hereby incorporated by reference in its entirety, for all purposes.

### BACKGROUND

The present disclosure uses the term “cognitive load” to describe an amount of processing resources that are used to perform a task (see also Oviatt, S. (2006). Human-Centered Design Meets Cognitive Load Theory: Designing Interfaces That Help People Think. In Proceedings of the 14th ACM International Conference on Multimedia, New York, N.Y., USA, 2006 (pp. 871-880). Association for Computing Machinery. https://doi.org/10.1145/1180639.1180831). In a system such as the human brain, processing resources may include concepts such as working memory and attention (Kirschner, P. A. (2002). Cognitive load theory: implications of cognitive load theory on the design of learning. Learning and Instruction, 12(1), 1-10. https://doi.org/https://doi.org/10.1016/S0959-4752(01)00014-7). Several interacting factors may influence the experienced cognitive load, including task complexity, instructional design, user abilities, and environments (Kirschner, 2002; Oviatt, 2006). Users may perform a variety of tasks throughout a day that consume varying portions of the available processing resources. For example, on average, resting without engaging in a defined task may consume less processing resources than gathering and querying information in a reading comprehension task (see also Gwizdka, J. (2010). Distribution of cognitive load in Web search. Journal of the American Society for Information Science and Technology, 61(11), 2167-2187. https://doi.org/https://doi.org/10.1002/asi.21385). Sustained periods of high cognitive load, experienced, for example, during the simultaneous performance of multiple, conflicting tasks, may result in an increased proneness to task-related errors (Laarni, J. (2021). Multitasking and interruption handling in control room operator work. In Human Factors in the Nuclear Industry (pp. 127-149). Woodhead Publishing. https://doi.org/10.1016/B978-0-08-102845-2.00007-7).

### SUMMARY

Aspects of the present disclosure include a method for predicting cognitive load. The method comprises: receiving, by a computing device, sensor measurements from each of two or more sensors, wherein the sensor measurements correspond to characteristics of a processing system; for each sensor of the two or more sensors: deriving, from the sensor measurements of the sensor, a first set of features predictive of the load of the processing system; generating, from the first set of features, a self-attention vector that characterizes each feature of the first set of features relative to each feature of the first set of features and each feature of a second set of features derived from sensor measurements of another sensor of the two or more sensors; and defining a feature vector from the first set of features and the self-attention vector that correspond to the sensor; generating, from the feature vector of at least one sensor of the two or more sensors, an input feature vector; generating, by a trained machine-learning model using the input feature vector, an indication of a load of the processing system; and outputting, by the computing device, the indication of the load of the processing system.

Another aspect of the present disclosure comprises a system comprising one or more processors and a non-transitory computer-readable media that includes instructions that when executed by the one or more processors, cause the one or more processors to perform the methods described above.

Another aspect of the present disclosure comprises a non-transitory computer-readable media that includes instructions that when executed by one or more processors, cause the one or more processors to perform the methods described above.

These illustrative embodiments are mentioned not to limit or define the disclosure, but to provide examples to aid understanding thereof. Additional embodiments are discussed in the Detailed Description, and further description is provided there.

### BRIEF DESCRIPTION OF THE DRAWINGS

Features, embodiments, and advantages of the present disclosure are better understood when the following Detailed Description is read with reference to the accompanying drawings.

FIG. 1 is a block diagram of a system for predicting cognitive load according to aspects of the present disclosure.

FIG. 2 is a block diagram illustrating corresponding levels of cognitive load according to aspects of the present disclosure.

FIG. 3 is a block diagram illustrating feature extraction from sensors measurements according to aspects of the present disclosure.

FIG. 4 is a diagram illustrating a self-attention neural network model according to aspects of the present disclosure.

FIG. 5 depicts an example flowchart of a process predicting a cognitive load of a user according to aspects of the present disclosure.

FIG. 6 illustrates a block diagram of an example device according to aspects of the present disclosure.

FIG. 7 is an example wearable watch device according to aspects of the present disclosure.

### DETAILED DESCRIPTION

Method and systems are disclosed herein for predicting cognitive load of a user. A cognitive load of a user may vary across tasks being performed by the user. Predicting cognitive load can lead to mitigation strategies that reduce or eliminate detrimental effects of sustained periods of high cognitive load (e.g., by determining when error rates are likely to exceed safety parameters, when to terminate performance of tasks, when to switch to low intensity tasks, when to switch to high intensity task, etc.). Predicting cognitive load may also help to identify cognitive issues such as detecting high cognitive load during tasks that should induce low levels of cognitive load.

In some examples, predicting cognitive load may include receiving sensor measurements from one or more sensors while a user is performing a task. The sensor measurements from each sensor may be pre-processed to extract a set of features (e.g., one set of features for each sensor type). The sets of features may be passed as into a machine-learning model to generate a prediction of the cognitive load of the user performing the task. The prediction may correspond to an integer, percentage, and/or category (e.g., low, medium, high, etc.) and include an indication of the task being performed by the user.

In some examples, predicting cognitive load may utilize a computing device (e.g., computer, mobile device, wearable devices, combinations thereof, and/or the like) that receive sensor measurements associated with a user. For example, the computing device may receive sensor measurements in the form of time series signals from an electroencephalogram (EEG) sensor, a photoplethysmogram (PPG) sensor, an inertial measurement unit (IMU), and/or an electrodermal activity (EDA) sensor. In some instances, sensor measurements may be received from other sensor types such as, but not limited to, electromyography (EMG), electrooculography (EOG), functional magnetic resonance imaging (fMRI), functional near-infrared spectroscopy (fNIRS), magnetoencephalography (MEG), inertial measurement unit (IMU), facial or body pose features, two-dimensional cameras, three-dimensional cameras, auditory responses, self-reporting surveys, or the like.

The computing device may process the sensor measurements to extract a set of features for each sensor. The measurements from each sensor may be pre-processed to reduce signal noise and/or other artifacts, to scale measurements (e.g., to reduce higher sampling rate sensors from being weighted higher due to the larger quantity of measurements, etc.) PPG sensor, and the like. Sensor measurements may be pre-processed based on a sensor type from which the sensor measurements originate (e.g., pre-processing IMU measurements may be different from pre-processing EEG measurements). Extracting features from the pre-processed sensor measurements may also be based on the corresponding sensor type.

An input feature vector may be generated from each set of features such that an input vector may be generated for each sensor. A trained machine-learning model may be executed using the input feature vector or the set of features of each sensor to generate a prediction of the cognitive load of the user. In some instances, feature selection may be performed on each set of features to determine the features from the set of features that will be included in the input feature vector. In some instances, the machine-learning model may be a support vector machine. In other instances, the machine-learning model may be a neural network, such as a convolutional neural network (in which the input vector may be represented in matrix form), or the like. In still yet other instances, the machine-learning model may a regression-based model (linear, logistic, etc.), decision tree, Naive Bayes, nearest neighbor, k-means, or any classification model.

Generating the input feature vector can include, for each sensor, executing feature projection (e.g., dimensionality reduction) for the set of features using a linear algorithm (e.g., principal component analysis) or non-linear algorithm (e.g., kernel or graph-based principal component analysis). A self-attention vector may be generated for the reduced set of features. The self-attention vector may include a score for each feature relative to other features in a set of features. The score indicates the relationship between a feature and another feature. The self-attention vector may be normalized (e.g., using a softmax algorithm, or the like). A self-attention vector may be integrated into the projected set of features to generate a self-attention feature vector (e.g., by taking the tensor product of the self-attention vector and the projected feature vector). The self-attention feature vector associated with each sensor may be aggregated (e.g., such as through a summation function or the like) to generate the input feature vector (associated with multiple sensors).

The trained machine-learning model may be executed using the input feature vector to generate the prediction of the cognitive load of the user. In some instances, one or more actions may be performed based on the prediction of the cognitive load such as, but not limited to, terminating the current task, switching to a new task, generating an alert (auditory or visual), modifying the instructional design of the task (e.g., by modifying a user interface, or the like) to modify the experienced cognitive load, or the like.

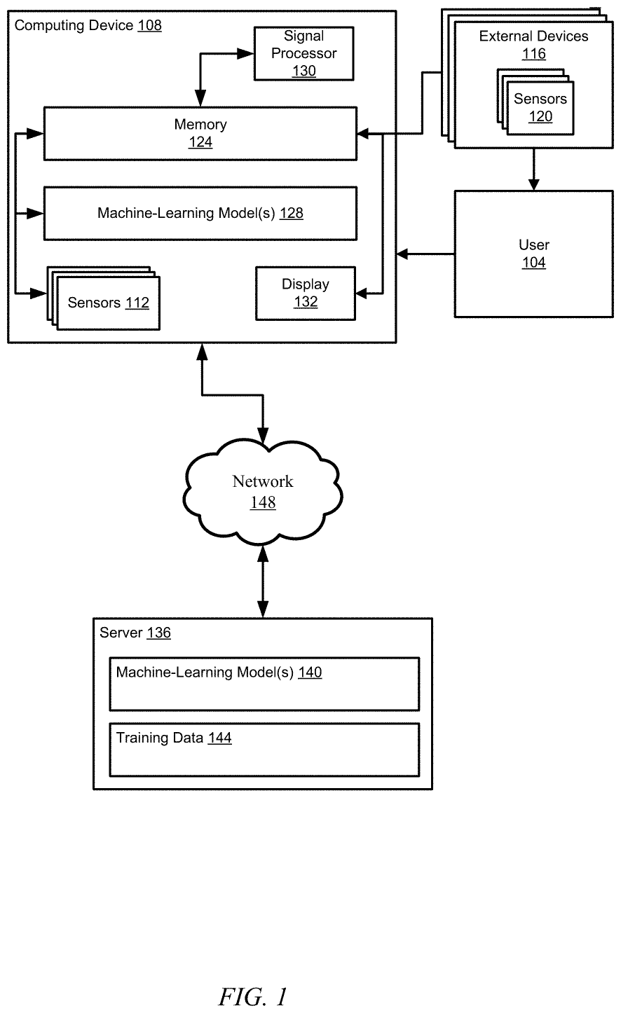

FIG. 1 is a block diagram of a system for predicting cognitive load according to aspects of the present disclosure. User 104 may perform tasks that consume an amount of available processing resources (referred to as “cognitive load” in the present disclosure). Different tasks may affect the experienced cognitive load differently. For instance, on average, resting without engaging in a defined task may consume less processing resources than gathering and querying information in a reading comprehension task. Computing device 108 may process sensor measurements to generate an estimated cognitive load, which may be availed to the user. Computing device 108 may be an electronic processing device (e.g., a computer, smartphone, smartwatch, smart audio devices, etc.) that processes sensor measurements that correspond to user 104. Computing device 108 may generate predictions of a current cognitive load of user 104 from the sensor measurements. In some instances, computing device 108 may modify the instructional design of the task performed by user 104 to modify the experienced cognitive load.

Computing device 108 may receive sensor measurements from internal sensors 108 of computing device 108. When computing device 108 is affixed to user 104 (e.g., in a pocket of user 104, be held or worn by user 104, etc.), the sensor measurements associated with computing device 108 may be imputed onto user 104 and become characteristics of user 104. For instance, internal sensors 112 may include an accelerometer that measures acceleration forces of computing device 108. When computing device 108 is affixed to user 104, acceleration measurements collected by the computing device may also correspond to an acceleration of user 104.

In some instances, computing device 108 may receive sensor measurements from one or more external devices 116 associated with user 104. External devices 116 may include devices configured to measure characteristics of user 104 using sensors 120. External device 116 may also receive sensor measurements from other devices. For example, an external device may be a wearable device (e.g., such as a smartwatch) that includes sensors 120 to measure characteristics of a wearer. External device 116 may receive sensor measurements from other external devices such as an EEG device that measures EEG signals. The wearable device may transmit sensor measurements from sensors 120 and sensor measurement received from the EEG device computing device 108. Examples of external devices 116 may include, but are not limited to, computing devices (e.g., similar to or different from computing device 104), wearable devices (e.g., smartwatches or the like), specialized devices configured to collect and store sensor measurements for transmission to computing device 104, or the like. Computing device 104 may receive sensor measurements (from both internal sensors 112 and sensor 120) in real time and store collected sensor measurements in memory 124.

Internal sensors 108 and sensors 120 may include sensors of any type. Examples of internal sensors 108 and sensors 120 include, but are not limited to, accelerometers, gyroscopes and magnetometers inside an IMU, thermometers, EEG sensors, microphones, EDA sensors (e.g., such as a galvanic skin response sensor, etc.), heart rate sensors (e.g., such as PPG sensors, etc.), and the like.

Computing device 104 may process sensor measurements received from sensors 112 and sensors 120 using signal processor 130. Computing device 108 may process each sensor measurements based on a sensor type of the sensor from which the sensor measurements originated. For instance, sensor measurements from an EDA sensor may be interpolated while sensor measurements from other sensors may not be interpolated. Processing of sensor measurements can include, but is not limited to, an interpolation process to add additional estimated sensor measurements for sensors with low sampling rates or missing measurements, filtering to reduce noise, epoching to define time windows for further processing, an artifact removal process (e.g., using wavelet thresholding or the like), combinations thereof, and the like. Once the sensor measurements are processed, a set of features may be extracted from the sensor measurements of each sensor. Computing device 108 may store the processed sensor measurements and the set of features may be extracted from the sensor measurements of each sensor in memory 124.

Computing device 108 can generate an input feature vector from the set of features of each sensor measurement. The input feature vector may include some or all of features from each set of features (e.g., corresponding to each of multiple sensors). In some instances, computing device 108 may select and/or weight features for inclusion into the input feature vector. Computing device 108 may use training data used to train machine-learning models 128 and/or metadata associated with previous predictions of the cognitive load to select and/or weight features from each set of features. For example, the metadata may include feature weights that are indicative of a degree in which a feature is a predictor of a cognitive load. Computing device 108 may then evaluate the feature weights to determine which features to include in the input feature vector and which features should be avoided. For example, features with a high feature weight (e.g., highly predictive) may be included, while features that have a low feature weight may not be included.

Computing device 108 may execute a trained machine-learning models 128 using the input feature vector to generate a prediction of the cognitive load of user 104. Machine-learning models may be trained using supervised, unsupervised, or semi-supervised learning. In some instance, machine-learning model 128 may be trained using historical sensor data collected from similar sensors to internal sensors 112 and sensors 120. For instance, sensor measurements may be collected from users using sensors of a computing device (such as computing device 108 or a computing device similar to computing device 108) and/or sensors of one or more external devices (such as external devices 116 or one or more external devices similar to external devices 116). The sensor measurements may be obtained while a user performs a defined task to induce different levels of cognitive load (e.g., resting without engaging in a defined task, a reading comprehension task, an item memorization task, or the like). Training data may be defined from the sensor measurements. The training data may be labeled (for supervised learning) with an indication of the task that was performed. In other instances, machine-learning models may be trained using generated data (e.g., such as manually or procedurally generated data).

Machine-learning models may be trained by computing device 108 or from a remote device such as server 136. When trained by computing device 108, the training data may be received from training data 144 of server 136. Server 136 may receive the training data (e.g., from computing device 108, external devices 116, one or more users, one or more other computing devices associated with users, one or more other external devices associated with users, one or more servers, users, or the like) or may generate the data (e.g., procedurally, or the like). Alternatively, machine-learning models may be trained remotely. For instance, machine-learning models 140 may be stored in server 136. Computing device may transmit a request for a trained machine-learning model over network 148 to server 136. If server 136 has trained a machine-learning model that corresponds to the model requested by computing device 108, then server 136 may transmit an instance of the requested, trained machine-learning model to computing device 108. If server 136 does not have a trained machine-learning model that corresponds to the model requested by computing device 108, then server 136 may train an untrained machine-learning model (of the particular requested model type) or obtain an instance of the requested, trained machine-learning model from another device. Server 136 may then transmit the instance of the requested, trained machine-learning model to computing device 108.

Computing device 108 may execute the trained machine-learning models using the input feature vector to generate the prediction of the current cognitive load of user 104. The prediction may be an image, integer, percentage (relative to a threshold value), a category (e.g., low, medium, high, etc.), an indication of the task being performed, or type of task being performed, or combinations thereof, or the like. Computing device 108 may generate predictions continuously (e.g., as sensor measurements are received), in predetermined time intervals, at preset times, upon detecting an event (e.g., user input, receiving a communication or push notification, upon indication that user 104 is performing a predetermined task or a new task, receiving a predetermined quantity of sensor measurements, detecting one or more sensor measurements that are greater than a threshold, combinations thereof, or the like), combinations thereof, or the like. Computing device 108 may store the prediction of the current task in association with a timestamp (e.g., time and/or date) in memory 124. Computing device 108 may present the prediction of the current cognitive load through display 132. Alternatively, or additionally, computing device 108 may transmit the prediction of the current task to server 136 (e.g., over network 148) or to an external device 116.

In some instances, computing device 108 may present a warning to user 104, another user, server 136, or the like when the predicted cognitive load is too high, too low, or when the predicted cognitive load remains high for a threshold time interval. For instance, if the predicted cognitive load is high for greater than a threshold time interval, computing device 108 may present a visual alert (e.g., through display 132), a haptic alert, and/or an auditory alert. Alternatively, computing device 108 may transmit an alert communication (e.g., push notification, email, text message, or the like). The alert may indicate that the threshold time interval has been exceeded and provide an indication of the predicted cognitive load. The alert may also suggest that user 104 should terminate the high cognitive load task and perform a low or lower cognitive load task (e.g., such as resting without engaging in a defined task).

In some instances, computing device 108 may modify the instructional design of the task performed by user 104 to modify the experienced cognitive load or alert the user to complete a task. For example, if the task involves an interaction with a technical system (e.g., operation of a user interface, interaction with a remote device, etc.), computing device 108 may modify user interfaces which may modify the experienced cognitive load during the performance of the task. Alternatively, computing device 108 may terminate the current task and present user 104 with a low or lower cognitive load task or no task at all. Alternatively still, computing device may present user 104 with a range of low or lower cognitive load tasks. In another example, if the cognitive load is predicted as low when the user is performing a task that should induce a medium cognitive load, a notification may be generated.

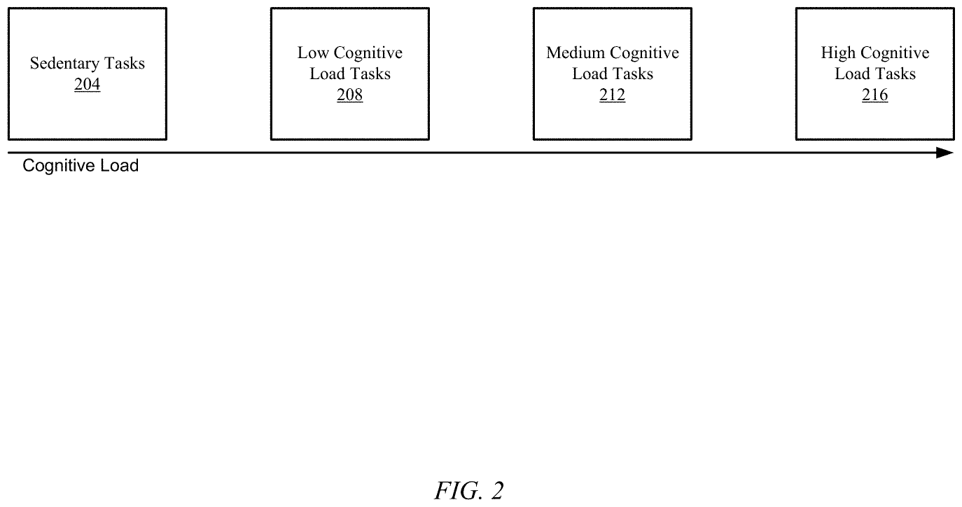

FIG. 2 is a block diagram illustrating example task types and corresponding levels of cognitive load according to aspects of the present disclosure. The present disclosure uses the term “cognitive load” to describe the amount of processing resources that are used to perform a task and refers to low, medium, or high “cognitive load tasks” to describe tasks that may induce varying levels of cognitive load. The cognitive load induced during the performance of a task may vary across tasks and users: Several interacting factors may influence the experienced cognitive load, including task complexity, instructional design, user abilities, and environments (Kirschner, 2002; Oviatt, 2006).

While at a sedentary task 204 (e.g., resting without engaging in a defined task), the cognitive load of a user may be lowest. A low cognitive load task 208 may increase cognitive load, therefore a user's cognitive load may be higher than during the performance of a sedentary task 204. For example, browsing and reading social media posts without a further defined task goal could be considered as a low cognitive load task relative to gathering and querying information in a reading comprehension task (see also Gwizdka, 2010). The experienced cognitive load may vary, for example, based on the specific task being performed, instructional design, user abilities, and environments (Kirschner, 2002; Oviatt, 2006).

Medium cognitive load tasks 212 may induce a medium-to-high cognitive load. Gathering and querying information in a reading comprehension task could be considered as a medium cognitive load task. A user may be asked to gather and process information, relate it to previously learned information, and answer questions based on the processed contents (see also Gwizdka, 2010). Medium cognitive load tasks 212 may induce higher cognitive load than low cognitive load tasks 208 and sedentary tasks 204. Medium cognitive load tasks 212 may induce different levels of cognitive load depending on the specific task being performed, instructional design, user abilities, and environments (Kirschner, 2002; Oviatt, 2006).

High cognitive load tasks 216 may induce a medium-to-high cognitive load, for example, as a result of simultaneously performing multiple, conflicting tasks at a given time (Laarni, 2021). High cognitive load tasks 216 may induce a higher cognitive load than medium cognitive load tasks 212.

The tasks are presented in increasing order in which the tasks may induce levels of cognitive load. For instance, sedentary tasks 204 may induce the least cognitive load, followed by low cognitive load tasks 208, then medium cognitive load tasks 212, and then high cognitive load tasks 216. The induced cognitive load of low cognitive load tasks 208, medium cognitive load tasks 212, and high cognitive load tasks 216 may vary, for example, based on the specific task being performed, instructional design, user abilities, and environments (Kirschner, 2002; Oviatt, 2006).

The tasks depicted are examples of tasks that may induce varying levels of cognitive load during the performance of the tasks. Other tasks (not shown) may be used for training the machine-learning models. Predicting levels of cognitive load during performance of tasks (those shown and those not shown) may be extended to any task.

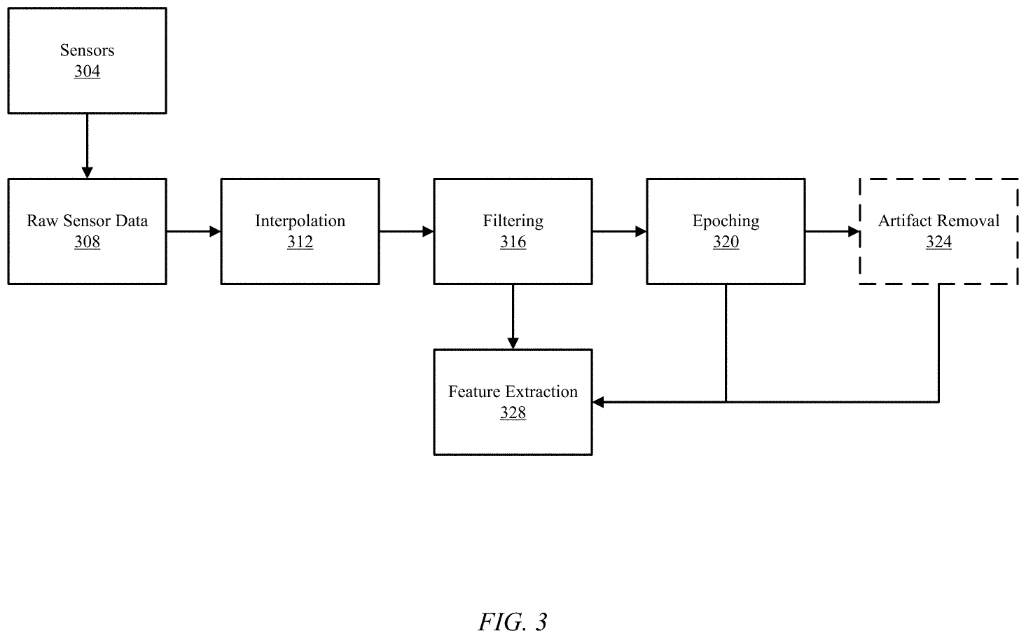

FIG. 3 is a block diagram illustrating feature extraction from sensor measurements according to aspects of the present disclosure. Sensor measurements associated with a user may be processed by a computing device to derive features for an input feature vector. The sensor measurements may be processed in one or more processing stages. The sensor type of each sensor may determine the processing stages executed by the computing device. For instance, the computing device may execute more processing stages when processing sensor measurements from some sensor types than sensor measurements from other sensor types. The processing stages may include, but are not limited to, interpolation 312, filtering 316, epoching 320, artifact removal 324, and the like. Though described in a particular order, the processing stages may be performed in any order including orders not shown.

The computing device may receive sensor measurements from sensors 304 (e.g., such as internal sensors 112 and/or sensors 120 of FIG. 1). The sensor measurements can include, for example, an EDA sensor, an IMU, a PPG sensor, an EEG sensor, and/or the like. Sensor measurements may be received as time series data in which each sensor measurement may be associated with a time in which that measurement was generated (by a corresponding sensor) or received. The quantity of sensor measurements received may be based on the number of channels of the sensor type (e.g., each may generate a sensor measurement) and the sampling rate. For instance, EDA may include two or more channels and have a low sampling rate, while EEG sensors can include between 1 and 32 channels and have a high sampling rate.

Processing the sensor measurements can include executing an interpolation stage 312. During this stage, the computing device may determine whether the quantity of received sensor measurements over a time interval is greater than a threshold. If the computing device does not receive a threshold quantity of sensor measurements from a particular sensor (e.g., due to a low sampling rate, noise, corrupted sensor measurements, etc.), the corrupted sensor measurements may end up being weighted more heavily in predicting cognitive load. This may result in an inaccurate prediction. The computing device may determine if the quantity of sensor measurements of a particular sensor type is greater than the quantity threshold. If not, the computing device may execute an interpolation process to generate estimated sensor measurements based on the received sensor measurements. The interpolation process may use a linear algorithm, function algorithm, Gaussian algorithm, or the like to generate the estimated sensor measurements.

The computing device may define a predetermined quantity of sensor measurements per sensor type that may represent a minimum quantity of sensor measurements that are to be received during a measurement time interval. The predetermined quantity may be based on the training data used to train the machine-learning model, previously generated predictions of the cognitive load, or the like. The sensor measurements received during the measurement time interval may be interpolated if the sensor measurements are not greater than the predetermined quantity of sensor measurements.

The computing device may then execute a filtering stage 316, in which the sensor measurements may be filtered. Filtering the sensor measurements may remove ranges of the sensor measurements that may be the result of signal noise. Filtering sensor measurements may include transforming the sensor measurements from the time domain into a frequency domain (e.g., using a Fourier transform or the like). The sensor measurements may then be filtered by a bandpass filter configured according to sensor type. For instance, a bandpass filter for EDA sensor measurements may filter frequencies in 0-1 Hz, 0-3 Hz, 0-5 Hz, or 0-x where x may be selected by as user. IMU sensor measurements may filter frequencies in 0-5 Hz, 0-10 Hz, 0-15 Hz, or 0-x where x may be selected by as user. PPG sensors measurements may filter frequencies in 0-3 Hz, 0-5 Hz, 0-8 Hz, or 0-x where x may be selected by as user. EEG signal measurements filter frequencies in 1-30 Hz, 1-55 Hz, 1-80 Hz, or 1-x where x may be selected by as user.

The computing device may then execute an epoching stage 320, in which the filtered sensor measurements are defined as multiple discrete sets of sensor measurements. The computing device may define a time window (e.g., an epoch) which may be used to divide the filtered sensor measurements into a series of time-windowed sets (e.g., each time-windowed set includes the sensor measurements collected within the time window). In some instances, the length of the time window may be based on the corresponding sensor type. In other instances, the length of the time window may be the same for each sensor type, which may be of a predetermined length (e.g., 15 seconds, 30 seconds, 45 seconds, or any time set by user input). The time interval may be static (e.g., each subsequent time interval does not overlap with previous time interval) or sliding (e.g., each subsequent time interval may overlap with the previous time interval). The sliding window may include an iteration value that indicates a rate at which the sliding window increments. An iteration value of 1 second indicates that a 30 second time window (e.g., T=0-30) will increment every second such that the next sliding window will include sensor measurements collected between T=1-31. The iteration value may be predetermined or based on user input.

In some instances, the time-windowed set may include artifacts (e.g., noise, outliers, measurements corresponding to an event not of interest, etc.). For example, EEG sensor measurements may include artifacts resulting from eye-blinks. The computing device may determine if artifact removal is needed for each time-windowed set. If the computing device determines that artifact removal is not needed, then the process continues to block 328 where features may be extracted. If the computing device determines that artifact removal is needed, then the process continues to artifact removal 324. In some instances, the artifact removal 324 may be applied to EEG sensor measurements. In other instances, artifact removal 324 may be applied to sensor measurements of each sensor type. In still yet other instances, the computing device may determine whether to apply artifact removal 324 based a threshold (e.g., based on sensor measurements from a time-windowed set deviating from an average by more than a threshold amount, etc.).

Artifact removal 324 may remove sensor measurements that are the result of noise, an event not of interest, outliers, or the like. The artifact-removal stage may execute a wavelet thresholding process to remove artifacts. Wavelet thresholding may include executing a wavelet transform on each time-windowed set. A predetermined threshold may be applied to the transformed time window. The predetermined threshold may cause the transformed values that are less than the predetermined threshold to be set to zero. Alternatively, the predetermined threshold may cause the transformed values that are greater than the predetermined threshold to be set to zero. The predetermined threshold may be based on the sensor type, user input, or the like. An inverse wavelet transform may then be applied to the remaining transformed values of the transformed time-windowed set. The result of applying the wavelet thresholding is a removal of artifacts in the sensor measurements.

Feature extraction 328 may include deriving features from each time-windowed set. The features extracted from each time-windowed set may be based on the corresponding sensor type. For instance, for the EDA sensor, the computing device may extract a feature corresponding to an amplitude of each peak in the time-windowed set and a feature indicating a number of peaks in the time-windowed set. For the IMU, the computing device may extract a feature for the mean and a feature for the standard deviation of the acceleration in the x-axis, a feature for the mean and a feature for the standard deviation of the acceleration in the y-axis, and/or a feature for the mean and a feature for the standard deviation of the acceleration in the z-axis.

For the PPG sensor, the computing device may extract time-domain features, frequency-domain features, and/or non-linear features. The time-domain features can include, but are not limited to, heartrate variability, heart rate, the standard deviation of the normal-to-normal intervals (SDNN), the standard deviation of the average normal-to-normal intervals (SDANN), the root mean square of the success difference (RMSSD), the heart rate variability triangular index (HTI), a number of adjacent number-to-number intervals that differ by more than 50 ms (nn50), a percentage of adjacent number-to-number intervals that differ by more than 50 ms (pNN50), and the like. The frequency domain features can include a feature indicative of the power values and a feature corresponding to the peak values extracted from three frequency bands: very low frequencies (e.g., 0.0033-0.04 Hz), low frequencies (e.g., 0.04-0.15 Hz), and/or high frequencies (e.g., 0.15-0.4 Hz). The frequency domain features also include a feature corresponding to a ratio of the low frequency power to the high frequency power. The non-linear features can include, but are not limited to, the area under the ellipse (e.g., the plotted time-windowed sensor measurements), approximate entropy, sample entropy, detrended fluctuation analysis (of short-term fluctuations and/or long-term fluctuations), correlation dimensions, Poincare plot standard deviation perpendicular to the line of identity (SD-1), Poincare plot standard deviation along to the line of identity (SD-2), ratio of SD-1 to SD-2, or the like.

For EEG sensors, the computing device may extract a feature for measured band-power in frequency bands such as Gamma, Beta, Alpha, Theta, and Delta bands. In some instances, feature extraction for EEG sensors may use a power spectral density analysis such as a Fourier power spectral density analysis. Since EEG sensors may include multiple channels (e.g., 1 or 32 channels), feature extraction 328 may include a feature for each frequency band for each channel for either 5 (frequency bands)*1 (channels)=5 features or 5 (frequency bands)*32 (channels)=160 features.

In some instances, a feature vector may be defined from the features from one or more sensor types. The one or more sensor types may be selected based on previous predictions by a machine-learning model, user input, or the like. In some instances, the feature vector may include features from an EEG sensor. In other instances, the feature vector may include features from an EEG sensor and features from an EDA sensor, IMU, and/or PPG sensor. The feature vector may be passed as input into a trained machine-learning model. For example, the machine-learning model may be a support vector machine or a convolutional neural network. The machine-learning model may generate a prediction indicative of the cognitive load of a user.

In some instances, the feature vector of each sensor type may be used to predict cognitive load. For instance, the feature vectors may be passed as input into a machine-learning model to predict the cognitive load of the user. The machine-learning model may be a convolutional neural network, support vector machine, regression model (e.g., linear, logarithmic, etc.), k-means, nearest neighbor, or the like.

### EXPERIMENTAL RESULTS

Predicting cognitive load during the performance of tasks was tested to determine the accuracy of the prediction given the use of different sensors. Sensor measurements were obtained while a user was performing three tasks: resting without engaging in a defined task, browsing and reading social media posts without a further defined goal, and a reading comprehension task. The sensor measurements were captured from a (4 channel) EEG sensor, a (29 channel) EEG sensor, a two channel EDA sensor, an IMU sensor, and a PPG sensor. The sensors were pre-processed to extract a set of features for each sensor (e.g., as described above). Different combinations of sensors were utilized as input to determine the accuracy of the machine-learning models and to determine which sensor or combination of sensors caused the machine-learning models to generate the most accurate prediction of the cognitive load of the user. A support vector machine (i.e., a linear classifier) and a convolutional neural network were used as machine-learning models. The machine-learning models were trained using sensor measurements collected from 15 users. Accuracies were computed using a leave-one-subject-out cross validation.

A (29 channel) EEG sensor was used to capture sensor measurements while a user was resting, and at a different time, reading (e.g., reading a peer-reviewed academic paper). The convolutional neural network estimated the cognitive load of the user during the performance of the resting task versus the reading task with a 96% (±4%) accuracy. The support vector machine estimated the cognitive load of the user during the performance the two tasks with a 94% (±5%) accuracy. Another test was performed in which the (29 channel) EEG sensor was used to capture sensor measurements while the user was performing a low cognitive load task and a reading comprehension task. The convolutional neural network estimated the cognitive load of the user during the performance of the low cognitive load task (e.g., browsing and reading social media posts without a further defined goal) versus a reading comprehension task (e.g., reading a peer-reviewed academic paper) with a 79% (±12%) accuracy. The support vector machine estimated the cognitive load of the user during the performance the two tasks with an 83% (±12%) accuracy.

The support vector machine was tested to determine which sensors would generate the most accurate predictions. Sensor measurements were received while a user was performing a resting task (with their eyes closed) and a reading comprehension task (e.g., reading a peer-reviewed academic paper). The support vector machine, using the (29 channel) EEG sensor, had an overall accuracy of 94% (±4%) (as previously shown). The support vector machine, using the (4 channel) EEG sensor, had an overall accuracy of 89% (±7%). The support vector machine, using heart rate variability (HRV) (e.g., derived from the PPG sensor), had an overall accuracy of 65% (±12%). The support vector machine, using the IMU, had an overall accuracy of 75% (±11%).

The support vector machine was then tested to determine which combination of sensors would generate the most accurate predictions. Sensor measurements were received while a user was performing a resting task (with their eyes closed) and a reading comprehension task (e.g., reading a peer-reviewed academic paper). The support vector machine, using the (29 channel) EEG sensor, PPG sensor, IMU sensor, and EDA sensor, had an overall accuracy of 94% (±5%). The support vector machine, using the (4 channel) EEG sensor, PPG sensor, IMU sensor, and EDA sensor, had an overall accuracy of 89% (±5%). The support vector machine, using the PPG sensor, IMU sensor, and EDA sensor, had an overall accuracy of 75% (±10%). Sensor measurements were then received while a user was performing a resting task (with their eyes opened) and a reading comprehension task. The support vector machine, using the (29 channel) EEG sensor, had an overall accuracy of 80% (±10%). The support vector machine, using the (29 channel) EEG sensor, PPG sensor, IMU sensor, and EDA sensor, had an overall accuracy of 83% (±10%). The support vector machine, using the (4 channel) EEG sensor, had an overall accuracy of 74% (±10%). The support vector machine, using the (4 channel) EEG sensor, PPG sensor, IMU sensor, and EDA sensor, had an overall accuracy of 80% (±10%). The support vector machine, using the PPG sensor, IMU sensor, and EDA sensor, had an overall accuracy of 77% (±12%).

Sensor measurements were received while a user was performing a reading comprehension task (e.g., reading a peer-reviewed academic paper) and a low cognitive load task (e.g., browsing and reading social media posts without a further defined goal). The support vector machine, using the (29 channel) EEG sensor, had an overall accuracy of 83% (±12%). The support vector machine, using the (29 channel) EEG sensor, PPG sensor, IMU sensor, and EDA sensor, had an overall accuracy of 97% (±2%). The support vector machine, using the (4 channel) EEG sensor, had an overall accuracy of 81% (±10%). The support vector machine, using the (4 channel) EEG sensor, PPG sensor, IMU sensor, and EDA sensor, had an overall accuracy of 97% (±2%). The support vector machine, using the PPG sensor, IMU sensor, and EDA sensor, had an overall accuracy of 98% (±2%).

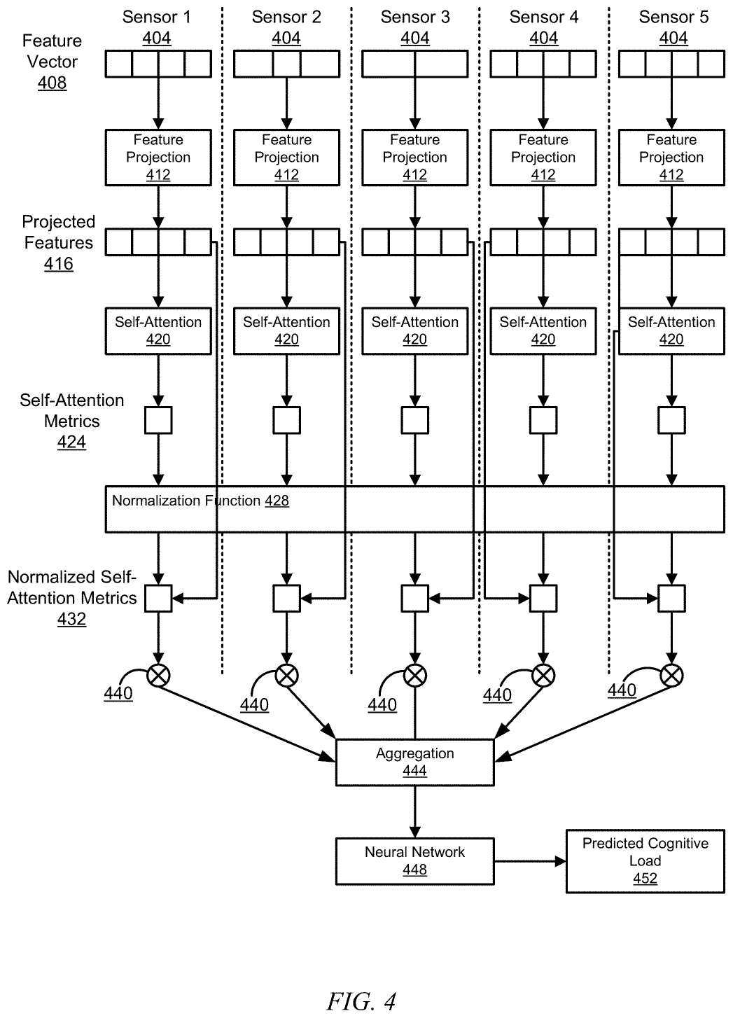

FIG. 4 is a diagram illustrating a self-attention neural network model according to aspects of the present disclosure. A feature vector may be derived from the features extracted from the sensor measurements of each sensor type 404. Examples of sensor types include, but are not limit to, an EDA sensor, an EEG sensor, a PPG sensor, an IMU, and the like. The feature vectors may be processed to generate an input feature vector. The trained neural network may be executed using the input feature vector to generate a prediction of a cognitive load. Feature vectors 408 (including features extracted from sensor measurements as described in connection to FIG. 3) from each sensor type 404 may be passed to feature projection module 412.

Feature projection 412 may reduce the features (e.g., from a high-dimensional space to a lower-dimensional space) while retaining the context of the features. For example, the feature vectors of some sensors may include sparse values, redundant values, too many values relative to other sensor types, etc. Processing features with a large quantity of values relative to features with few values may cause the neural network to favor the features with the large quantity of values. This may skew the accuracy of the neural network. Feature projection 412 reduces the quantity of features without eliminating the information provided by the features. Feature projection 412 may use a principal component analysis (PCA), kernel PCA, linear discriminant analysis, or the like.

Projected feature vector 416 output from feature projection 412 may be passed to self-attention module 420. Self-attention 420 may include scores for each feature. The scores include a score that corresponds to the degree in which a relationship exists between a feature and itself and a score that corresponds to the degree in which a relationship exists between a feature and each other feature in the set of features. This enables the machine-learning model to process features with a context of the relationship between features.

Generating the attention scores includes generating three weight matrices, each being initialized using a random distribution such as a Gaussian distribution, a Xavier initialization, or the like. In some instances, the weight matrix may be updated by the output from the machine-learning model.

For each feature in the feature vector:
A key matrix, a query matrix, and a value matrix may be generated by multiplying a weight matrix by the feature.
A score may be defined for each feature in the feature vector by calculating the dot product of the query matrix of this feature and the key matrix of each feature in the feature vector (including this feature).
A softmax function may be executed on the scores. The softmax function may normalize the scores such that the sum of all of the scores is equal to 1.
The score of each feature may then be multiplied by the value matrix of that feature (including this feature) to generate a score matrix. The dimensions of the score matrix may be based on the dimensions of the value matrix. In some instance, the dimensions of the value matrix may be predetermined (e.g., by manipulation of the weight matrix that defined the value matrix) to cause the score matrix to be of the same predetermined dimension as the value matrix. For instance, the dimensions of the value matrix may be equal to the quantity of features of the feature vector. This may cause the score matrix to include a score value for each feature (including this feature). A score value may be included that indicates a degree of relatedness between a feature and itself. The degree of relatedness between a feature and each other feature in the feature vector may be represented by a separate score value.
Each score matrix may be added together to form a self-attention vector of this feature.
The process then moves to the next feature in the feature vector and repeats until a self-attention vector is generated for each feature of the feature vector and there are no more features to iterate over.
The self-attention vector of each feature of the feature vector may be aggregated to form a single self-attention vector 424.

The process then continues by executing a normalization function 428 on the self-attention vector. The normalization function may be a softmax function that modifies the values of the self-attention vector to sum to 1. The relative magnitude of each value may be retained (e.g., a value with a large original value may assigned a new value that is close to 1 and a value with a small original value may be assign a new value that is close to zero, etc.).

The self-attention vector and the feature vector may be aggregated into a single vector by deriving the tensor product 440 of the attention metrics and the feature vector. The tensor product of each sensor may aggregate at 444. In some instances, aggregating may include a adding the tensor products together to form an input feature vector configured to be the input to neural network 448.

Neural network 448 may be trained using unsupervised, supervised, or semi-supervised learning. Sensor measurements associated with users may be received. During supervised learning, at least some of the sensor measurements may be labeled. The label may correspond to the cognitive load (e.g., a category, a percentage, an integer, or the like) of the user and/or a task that was being performed when the sensor measurements were collected. In some instances, some or all of the labels may be generated by neural network 448 or another machine-learning model.

Once trained, neural network 448 may be configured to generate predictions of the cognitive load and/or task being performed by a user using the input feature vector. In some instances, neural network 448 may output metadata associated with the prediction and the features of the input feature vector. Self-attention 420 may use the metadata to improve the three weight matrices, which may improve the self-attention vector and subsequent predictions by the neural network.

The self-attention neural network (FIG. 4) was tested relative to the support vector machine to determine a difference in accuracy (if any) between the accuracy of self-attention neural network and the support vector machine. Sensor measurements were collected from a 4 channel EEG sensor, a PPG sensor, a IMU sensor, and a GSU sensor while a user performed a resting task (e.g., with their eyes opened or closed) and a reading comprehension task (e.g., reading a peer-reviewed academic paper). The support vector machine had an overall accuracy of 81% (±7%). The self-attention neural network had an overall accuracy of 83% (±5%). The baseline accuracy was 50%.

A cross-validation (e.g., leave-one-subject-out or LOO) was performed on one or more missing sensor types. In a first test, each observation was based on the 4 channel EEG sensor being present and the IMU sensor, PPG sensor, and the EDA sensor being missing. The support vector machine had an overall accuracy of 77% (±10%). The self-attention neural network had an overall accuracy of 77% (±10%). The baseline accuracy of random guessing was 50%.

In a second test, each observation was based on the IMU sensor, PPG sensor, and the EDA sensor being missing. The support vector machine had an overall accuracy of 75% (±10%). The self-attention neural network had an overall accuracy of 71% (±10%). The baseline accuracy was 50%.

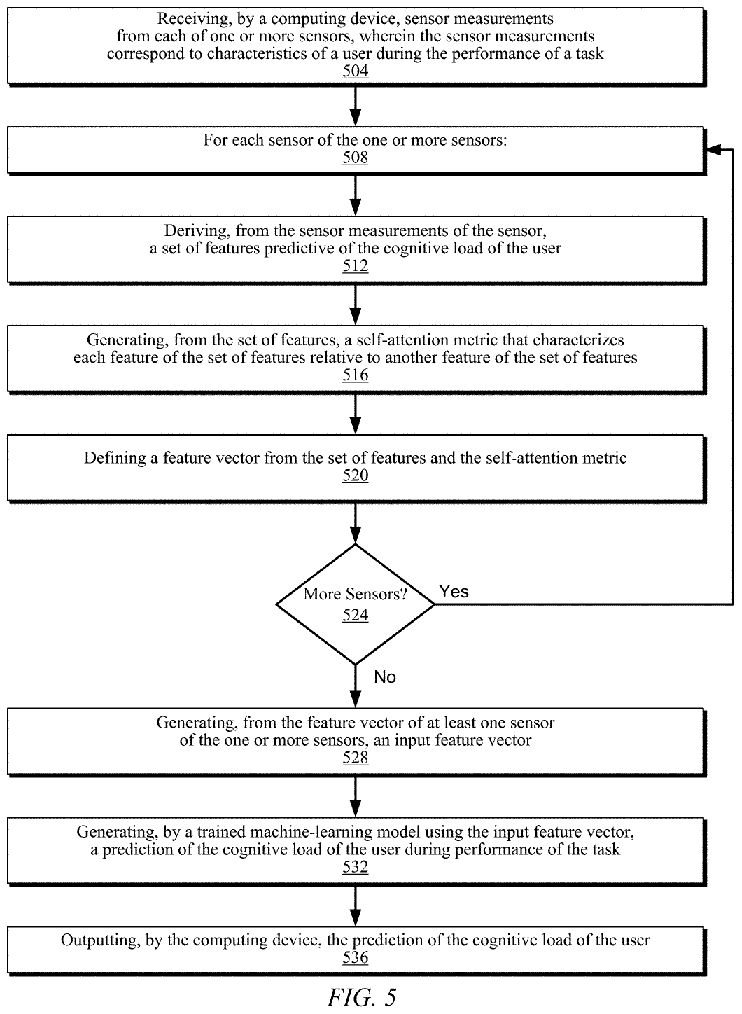

FIG. 5 depicts an example flowchart of a process predicting the cognitive load of a user according to aspects of the present disclosure. At block 504, a computing device may receive sensor measurements from each of one or more sensors. The sensor measurements may correspond to characteristics of a user during the performance of a task. The computing device may be a device operated by the user such as, but not limited to, a mobile device or a wearable device, or the like. The computing device may receive the sensor measurements from sensors internal to the computing device. For instance, the computing device may receive sensor measurement from an internal sensor such as an accelerometer or IMU sensor. The computing device may also receive sensor measurements from one or more external devices. For instance, the computing device may receive some sensor measurements from a wearable device (e.g., worn by the user) that includes an EDA sensor or an EEG sensor. Examples of sensors from which sensor measurements may be received include, but are not limited to, accelerometers, gyroscopes, inertial measurement units, thermometers, IMU, microphones, EMG sensors, EEG sensors, EDA sensors, heart rate sensors (e.g., such as photoplethysmogram sensors, etc.), fNIRS sensors, infrared sensors, (two and/or three dimensional) cameras and the like.

At block 508, the computing device iterates over each sensor of the one or more sensors to generate a feature vector for each sensor. For each sensor, the computing device derives a first set of features predictive of cognitive load (at block 512). Features may be extracted from the set of sensor measurements based on the sensor type. The computing device may process the set of sensor measurements by interpolating the sensor measurements, filtering the sensor measurements based on a frequency selected according to the sensor type, epoching (e.g., generating time-windowed sets of sensor measurements), artifact removal (e.g., using wavelet thresholding or the like), and extracting the set of features as described in connect to FIG. 3.

In some instances, feature projections may be performed on the first set of features. For instance, the computing device may determine that feature projection is needed by analyzing the set of features. If the set of features is sparse or includes redundant values, the computing device may perform feature projection. Alternatively, if the quantity of set of features from a first sensor is greater than the quantity of the set of features from a second sensor by more than a threshold, then computing device may perform feature projection. Feature projection may reduce the features in the set of features (e.g., from a high-dimensional space to a lower-dimensional space). For instance, a feature set including sparse values may be reduced by reducing the sparse values (e.g., such as zeros or baseline values) and retaining the non-sparse values. Feature projection may be performed using a principal component analysis (PCA), kernel PCA, linear discriminant analysis, or the like on the set of features.

At block 516, the computing device then generates a self-attention vector for each feature of the set of features. The self-attention vector characterizes each feature of the set of features relative to another feature of the set of features. The self-attention vector may represent a weight that corresponds to the relationship between two features. When features are processed to generate predictions, the self-attention vector leverages the relatedness of the features to improve predictions. For instance, the set of features may include features across a time interval. The first feature and the last feature in the set of features may not appear related due to the temporal distance between the first feature and the last feature in the set.

The self-attention vector can define relatedness (e.g., a measure of how related the first feature may be to the last feature). As the first feature is being processed by a machine-learning model (such as a neural network, or the like), the machine-learning model may take advantage of relatedness to the last feature. In some instances, the relatedness may correspond to a range in which the value of a feature is dependent on the value of another feature or vice versa. For instance, if there is no relatedness between two features, then the value of one feature does not affect the value of another feature. If there is some relatedness between two features, then the value of one feature may affect the value of another feature. If there is a high degree of relatedness between two features, then the value of one feature may depend on the value of another feature.

In some instances, a softmax function may be executed on the self-attention vector of the set of features to normalize the self-attention vectors. The softmax function may reduce the values of self-attention vector such that the values sum to one.

At block 520, a feature vector may be defined from the set of features and the self-attention vector. In some instances, the feature vector may be generated by combining the set of features with the self-attention vector. For instance, the feature vector may be defined from the tensor product of the set of features and the self-attention vector.

At block 524, it is determined whether there are sensor measurements from a different sensor to process. If there are sensor measurements from another sensor, the process returns to block 508, where a next sensor is selected and blocks 512-520 are repeated to generate a feature vector from the sensor measurements of the new sensor. The processes of 508-524 may be repeated any number of times until a feature vector has been generated for each sensor of the one or more sensors. If it is determined that there are no additional sensors, then the process continues at block 528.

At block 528, an input feature vector may be generated. The input feature vector may be generated by aggregating the features vectors of each sensor. For instance, the feature vector from each sensor may be summed up to form the input feature vector.

At block 532, a trained neural network may be executed using the input feature vector to generate a prediction of the cognitive load of the user during performance of a task. The input feature vector incorporates the self-attention vector of the feature vectors to enable the trained neural network to leverage the relationships between features. The trained neural network may generate predictions that are more accurate by taking the feature relationships into account. The prediction may include an alphanumerical value such as an integer or a percentage, a category (e.g., low, medium, high, etc.), or the like that indicates the cognitive load of the user during performance of a task. Alternatively, or additionally, the prediction may include an indication of the task being performed by the user, which may indicate the cognitive load.

At block 536, the prediction of the cognitive load may be output. In some instances, the prediction of the cognitive load may be presented to the user (e.g., through a display of the computing device or by transmitting the prediction to a wearable device worn by the user). In other instances, the prediction of the task may be transmitted to a remote device. The remote device may store the prediction with an association to the user (e.g., in a user profile). The remote device may be operated by another user that may use the predictions to monitor the performance of the user.

In some instances, the computing device may execute one or more modifications to the current task based on the predicted cognitive load. The computing device may determine that the cognitive load of the user is greater than a threshold for a predetermined time interval. In response, the computing device may present an alert to the user (e.g., visual and/or auditory) indicating that the task should be halted or terminated. Alternatively, the computing device may halt or terminate the task. Alternatively still, the computing device modify the instructional design of the task. For instance, for a task involving operation of a device (such as the computing device) by the user, the computing device may modify interfaces of the device to modify the experienced cognitive load.

### EXAMPLE DEVICES

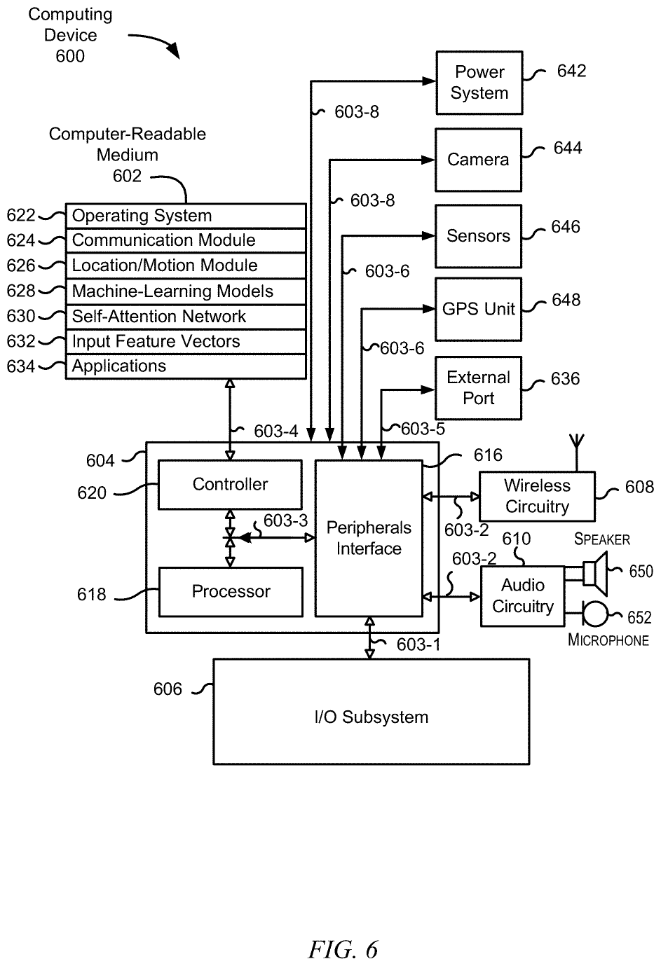

FIG. 6 is a block diagram of an example computing device 600. Computing device 600 generally includes computer-readable medium 602, a processing system 604, an Input/Output (I/O) subsystem 606, wireless circuitry 608, and audio circuitry 610 including speaker 650 and microphone 652. These components may be coupled by one or more communication buses or signal lines 603. Computing device 600 can be any portable electronic device, including a handheld computer, a tablet computer, a mobile phone, a smart watch, a smart audio device, a laptop computer, tablet device, media player, personal digital assistant (PDA), a key fob, a car key, an access card, a multi-function device, a portable gaming device, or the like, including a combination of two or more of these items.

It should be apparent that the architecture shown in FIG. 6 is only one example of an architecture for device 600, and that device 600 can have more or fewer components than shown, or a different configuration of components. The various components shown in FIG. 6 can be implemented in hardware, software, or a combination of both hardware and software, including one or more signal processing and/or application specific integrated circuits.

Wireless circuitry 608 is used to send and receive information over a wireless link or network to one or more other devices' conventional circuitry such as an antenna system, an RF transceiver, one or more amplifiers, a tuner, one or more oscillators, a digital signal processor, a CODEC chipset, memory, etc. Wireless circuitry 608 can use various protocols, e.g., as described herein.

Wireless circuitry 608 is coupled to processing system 604 via peripherals interface 616. Peripherals interface 616 can include conventional components for establishing and maintaining communication between peripherals and processing system 604. Voice and data information received by wireless circuitry 608 (e.g., in speech recognition or voice command applications) is sent to one or more processors 618 via peripherals interface 616. One or more processors 618 are configurable to process various data formats for one or more application programs 634 stored on medium 602.

Peripherals interface 616 couple the input and output peripherals of the device to processor 618 and computer-readable medium 602. One or more processors 618 communicate with computer-readable medium 602 via a controller 620. Computer-readable medium 602 can be any device or medium that can store code and/or data for use by one or more processors 618. Medium 602 can include a memory hierarchy, including cache, main memory and secondary memory.

Device 600 also includes a power system 642 for powering the various hardware components. Power system 642 can include a power management system, one or more power sources (e.g., battery, alternating current (AC)), a recharging system, a power failure detection circuit, a power converter or inverter, a power status indicator (e.g., a light emitting diode (LED)), and any other components typically associated with the generation, management and distribution of power in mobile devices.

In some embodiments, device 600 includes a camera 644. In some embodiments, device 600 includes sensors 646. Sensors can include accelerometers, a compass, a gyrometer, pressure sensors, audio sensors, light sensors, barometers, and the like. Sensors 646 may also include, but are not limited to, one or more of: an electroencephalogram sensor, a photoplethysmogram sensor, an inertial measurement unit (IMU), and/or an electrodermal activity sensor, electromyography, electrooculography, functional magnetic resonance imaging, functional near-infrared spectroscopy, magnetoencephalography, facial or body pose features, two-dimensional cameras, three-dimensional cameras, auditory responses, user input (e.g., response to self-reporting surveys, etc.), or the like. Sensors 646 can be used to sense location aspects, such as auditory or light signatures of a location.

In some embodiments, device 600 can include a GPS receiver, sometimes referred to as a GPS unit 648. A mobile device can use a satellite navigation system, such as the Global Positioning System (GPS), to obtain position information, timing information, altitude, or other navigation information. During operation, the GPS unit can receive signals from GPS satellites orbiting the Earth. The GPS unit analyzes the signals to make a transit time and distance estimation. The GPS unit can determine the current position (current location) of the mobile device. Based on these estimations, the mobile device can determine a location fix, altitude, and/or current speed. A location fix can be geographical coordinates such as latitudinal and longitudinal information.

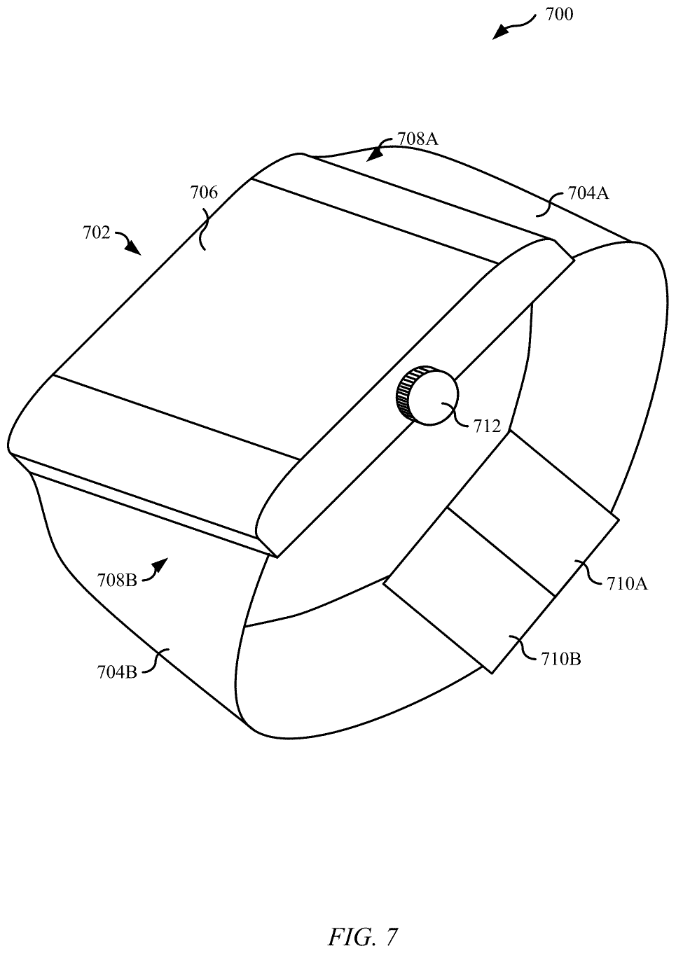

One or more processors 618 run various software components stored in medium 602 to perform various functions for device 600. In some embodiments, the software components include an operating system 622, a communication module (or set of instructions) 624, a location module (or set of instructions) 626, machine-learning models 628, a self-attention (or set of instructions) 630, an input feature vectors 632, and other applications (or set of instructions) 634, such as a car locator app and a navigation app. Machine-learning models 628 may include a set of instructions that correspond to a support vector machine, a neural network, a convolutional neural network, a self-attention neural network that when executed generates predictions of the current cognitive load of a user. Input feature vectors 632 may be derived from sensor measurements received from sensors of computing device 600 and/or from a wearable device (e.g., as described in FIG. 7) and from self-attention 630. Machine-learning models 628 may generate predictions using the input feature vector.

In some instances, computing device 600 may process sensor measurements captured by computing device 600 and/or received from one or more external devices (e.g., external sensors, another computing device, one or more wearable devices such as wearable device 700 of FIG. 7, smart audio devices, or the like) to generate cognitive load predictions (e.g., as described in FIG. 5). In other instances, computing device 600 may transmit sensor measurements captured by sensors 646 of computing device 600 to another device (e.g., another computing device 600, a wearable device, a database, a server, or the like). The other device may be configured to process the sensor measurements to generate cognitive load predictions or transmit the sensor measurements to device configured to generate cognitive load predictions.

Operating system 622 can be any suitable operating system, including iOS, Mac OS, Darwin, RTXC, LINUX, UNIX, OS X, WINDOWS, or an embedded operating system such as VxWorks. The operating system can include various procedures, sets of instructions, software components and/or drivers for controlling and managing general system tasks (e.g., memory management, storage device control, power management, etc.) and facilitates communication between various hardware and software components.

Communication module 624 facilitates communication with other devices over one or more external ports 636 or via wireless circuitry 608 and includes various software components for handling data received from wireless circuitry 608 and/or external port 636. External port 636 (e.g., USB, FireWire, a Lightning connector, a 60-pin connector, etc.) is adapted for coupling directly to other devices or indirectly over a network (e.g., the Internet, wireless LAN, etc.).

Location/motion module 626 can assist in determining the current position (e.g., coordinates or other geographic location identifier) and motion of device 600. Modern positioning systems include satellite-based positioning systems, such as Global Positioning System (GPS), cellular network positioning based on “cell IDs,” and Wi-Fi positioning technology based on a Wi-Fi networks. GPS also relies on the visibility of multiple satellites to determine a position estimate, which may not be visible (or have weak signals) indoors or in “urban canyons.” In some embodiments, location/motion module 626 receives data from GPS unit 648 and analyzes the signals to determine the current position of the mobile device. In some embodiments, location/motion module 626 can determine a current location using Wi-Fi or cellular location technology. For example, the location of the mobile device can be estimated using knowledge of nearby cell sites and/or Wi-Fi access points with knowledge also of their locations. Information identifying the Wi-Fi or cellular transmitter is received at wireless circuitry 608 and is passed to location/motion module 626. In some embodiments, the location module receives the one or more transmitter IDs. In some embodiments, a sequence of transmitter IDs can be compared with a reference database (e.g., Cell ID database, Wi-Fi reference database) that maps or correlates the transmitter IDs to position coordinates of corresponding transmitters, and computes estimated position coordinates for device 600 based on the position coordinates of the corresponding transmitters. Regardless of the specific location technology used, location/motion module 626 receives information from which a location fix can be derived, interprets that information, and returns location information, such as geographic coordinates, latitude/longitude, or other location fix data.

The one or more applications 634 on the mobile device can include any applications installed on the device 600, including without limitation, a browser, address book, contact list, email, instant messaging, word processing, keyboard emulation, widgets, JAVA-enabled applications, encryption, digital rights management, voice recognition, voice replication, a music player (which plays back recorded music stored in one or more files, such as MP3 or AAC files), etc.

There may be other modules or sets of instructions (not shown), such as a graphics module, a time module, etc. For example, the graphics module can include various conventional software components for rendering, animating and displaying graphical objects (including without limitation text, web pages, icons, digital images, animations and the like) on a display surface. In another example, a timer module can be a software timer. The timer module can also be implemented in hardware. The time module can maintain various timers for any number of events.

The I/O subsystem 606 can be coupled to a display system (not shown), which can be a touch-sensitive display. The display displays visual output to the user in a GUI. The visual output can include text, graphics, video, and any combination thereof. Some or all of the visual output can correspond to user-interface objects. A display can use LED (light emitting diode), LCD (liquid crystal display) technology, or LPD (light emitting polymer display) technology, although other display technologies can be used in other embodiments.

In some embodiments, I/O subsystem 606 can include a display and user input devices such as a keyboard, mouse, and/or track pad. In some embodiments, I/O subsystem 606 can include a touch-sensitive display. A touch-sensitive display can also accept input from the user based on haptic and/or tactile contact. In some embodiments, a touch-sensitive display forms a touch-sensitive surface that accepts user input. The touch-sensitive display/surface (along with any associated modules and/or sets of instructions in medium 602) detects contact (and any movement or release of the contact) on the touch-sensitive display and converts the detected contact into interaction with user-interface objects, such as one or more soft keys, that are displayed on the touch screen when the contact occurs. In some embodiments, a point of contact between the touch-sensitive display and the user corresponds to one or more digits of the user. The user can make contact with the touch-sensitive display using any suitable object or appendage, such as a stylus, pen, finger, and so forth. A touch-sensitive display surface can detect contact and any movement or release thereof using any suitable touch sensitivity technologies, including capacitive, resistive, infrared, and surface acoustic wave technologies, as well as other proximity sensor arrays or other elements for determining one or more points of contact with the touch-sensitive display.

Further, the I/O subsystem can be coupled to one or more other physical control devices (not shown), such as pushbuttons, keys, switches, rocker buttons, dials, slider switches, sticks, LEDs, etc., for controlling or performing various functions, such as power control, speaker volume control, ring tone loudness, keyboard input, scrolling, hold, menu, screen lock, clearing and ending communications and the like. In some embodiments, in addition to the touch screen, device 600 can include a touchpad (not shown) for activating or deactivating particular functions. In some embodiments, the touchpad is a touch-sensitive area of the device that, unlike the touch screen, does not display visual output. The touchpad can be a touch-sensitive surface that is separate from the touch-sensitive display or an extension of the touch-sensitive surface formed by the touch-sensitive display.

Aspects described herein may take the form of, be incorporated in, or operate with a suitable electronic device, e.g., companion devices, smart watch devices, or the like. One example of such a device is shown in FIG. 7 and takes the form of a wearable watch device. Alternative embodiments of suitable electronic devices include a mobile phone, a tablet computing device, a portable media player, smart audio devices (e.g., smart speakers, smart headphones, smart earbuds, or the like), and so on. Still other suitable electronic devices may include laptop/notebook computers, personal digital assistants, touch screens, input-sensitive pads or surfaces, and so on.

FIG. 7 shows a wearable watch device 700 according to some embodiments of the present invention. In this example, wearable device 700 is shown as a wristwatch-like device with a face portion 702 connected to straps 704A, 704B. In many embodiments, the electronic device may keep and display time, essentially functioning as a wristwatch among other things. Time may be displayed in an analog or digital format, depending on the device, its settings, and (in some cases) a user's preferences. Typically, time is displayed on a digital display stack forming part of the exterior of the device.

Face portion 702 can include, e.g., a touchscreen display 706 that can be appropriately sized depending on where on a user's person wearable device 700 is intended to be worn. A user can view information presented by wearable device 700 on touchscreen display 706 and provide input to wearable device 700 by touching touchscreen display 706. In some embodiments, touchscreen display 706 can occupy most or all of the front surface of face portion 702.

Straps 704A, 704B can be provided to allow wearable device 700 to be removably worn by a user, e.g., around the user's wrist, and secured thereto. In some embodiments, straps 704A, 704B can be made of any flexible material (e.g., fabrics, flexible plastics, leather, chains or flexibly interleaved plates or links made of metal or other rigid materials) and can be connected to face portion 702, e.g., by hinges. Alternatively, straps 704A, 704B can be made of a rigid material, with one or more hinges positioned at the junction of face 702 and proximal ends 708A, 708B of straps 704A, 704B and/or elsewhere along the lengths of straps 704A, 704B to allow a user to put on and take off wearable device 700. Different portions of straps 704A, 704B can be made of different materials; for instance, flexible or expandable sections can alternate with rigid sections. In some embodiments, one or both of straps 704A, 704B can include removable sections, allowing wearable device 700 to be resized to accommodate a particular user's wrist size. In some embodiments, straps 704A, 704B can be portions of a continuous strap member that runs behind or through face portion 702. Face portion 702 can be detachable from straps 704A, 704B; permanently attached to straps 704A, 704B; or integrally formed with straps 704A, 704B.

The distal ends of straps 704A, 704B opposite face portion 702 can provide complementary clasp members 710A, 710B that can be engaged with each other to secure the distal ends of straps 704A, 704B to each other, forming a closed loop. In this manner, device 700 can be secured to a user's person, e.g., around the user's wrist; clasp members 710A, 710B can be subsequently disengaged to facilitate removal of device 700 from the user's person. The design of clasp members 710A, 710B can be varied; in various embodiments, clasp members 710A, 710B can include buckles, magnetic clasps, mechanical clasps, snap closures, etc. In some embodiments, one or both of clasp members 710A, 710B can be movable along at least a portion of the length of corresponding strap 704A, 704B, allowing wearable device 700 to be resized to accommodate a particular user's wrist size.

Straps 704A, 704B can be two distinct segments, or they can be formed as a continuous band of an elastic material (including, e.g., elastic fabrics, expandable metal links, or a combination of elastic and inelastic sections), allowing wearable device 700 to be put on and taken off by stretching a band formed by straps 704A, 704B. In such embodiments, clasp members 710A, 710B can be omitted.

Straps 704A, 704B and/or clasp members 710A, 710B can include sensors that allow wearable device 700 to determine whether it is being worn at any given time. Wearable device 700 can operate differently depending on whether it is currently being worn or not. For example, wearable device 700 can inactivate various user interface and/or RF interface components when it is not being worn. In addition, in some embodiments, wearable device 700 can notify a companion device (e.g., a smartphone, a mobile device, a tablet device, a media player, a speaker, or other electronic devices) when a user puts on or takes off wearable device 700.

In various embodiments, wearable device 700 includes a rotary input such as a crown 712 (also referred to as digital crown throughout the specification). Crown 712 can be used to perform a variety of functions. In some embodiments, crown 712 provides rotation input for navigating content (e.g., zooming in and out of content, panning across content). In this example, crown 712 includes a plastic or metal crown body, preferably having conventional outer teeth. Typically, a pedestal made integral with the body of crown 715 is positioned and protrudes into face portion 702. Crown 712 may be fastened, either permanently or removably, to hardware associated with wearable device 700. Rotation of the crown (and/or a stem) may be sensed optically, electrically, magnetically, or mechanically. Further, in some embodiments the crown (and/or stem) may also move laterally, thereby providing a second type of input to the device.

Wearable device 700 may likewise include one or more buttons (not shown here). The button(s) may be depressed to provide yet another input to the device. In various embodiments, the button may be a dome switch, rocker switch, electrical contact, magnetic switch, and so on. In some embodiments the button may be waterproof or otherwise sealed against the environment.

Wearable device 700 may include one or more sensors for measuring characteristics of a user wearing wearable device 700. Examples of such sensors include, but are not limited to, an electroencephalogram sensor, a photoplethysmogram sensor, an inertial measurement unit (IMU), and/or an electrodermal activity sensor, electromyography, electrooculography, functional magnetic resonance imaging, functional near-infrared spectroscopy, magnetoencephalography, facial or body pose features, two-dimensional cameras, three-dimensional cameras, auditory responses, user input (e.g., response to self-reporting surveys, etc.), or the like.

In some instances, wearable device 700 may process sensor measurements captured by sensors of wearable device 700 and/or received from one or more external devices (e.g., external sensors, other wearable devices, computing device such as computing device 600 of FIG. 6, smart audio devices, or the like) to generate cognitive load predictions (e.g., as described in FIG. 5). In other instances, wearable device 700 may transmit sensor measurements captured by sensors of wearable device 700 to another device (e.g., computing device 600, another wearable device, a database, a server, or the like). The other device may be configured to process the sensor measurements to generate cognitive load predictions or transmit the sensor measurements to a device configured to generate cognitive load predictions.

It will be appreciated that wearable device 700 is illustrative and that variations and modifications are possible. For example, wearable device 700 can be implemented in any wearable article, including a watch, a bracelet, a necklace, a ring, a belt, a jacket, or the like. In some instances, wearable device 700 can be a clip-on device or pin-on device that has a clip or pin portion that attaches to the user's clothing. The interface portion (including, e.g., touchscreen display 706) can be attached to the clip or pin portion by a retractable cord, and a user can easily pull touchscreen display 706 into view for use without removing the clip or pin portion, then let go to return wearable device 700 to its resting location. Thus, a user can wear wearable device 700 in any convenient location.

Wearable device 700 can be implemented using electronic components disposed within face portion 702, straps 704A, 704B, and/or clasp members 710A, 710B.

In some embodiments, some or all of the operations described herein can be performed using an application executing on the user's device. Circuits, logic modules, processors, and/or other components may be configured to perform various operations described herein. Those skilled in the art will appreciate that, depending on implementation, such configuration can be accomplished through design, setup, interconnection, and/or programming of the particular components and that, again depending on implementation, a configured component might or might not be reconfigurable for a different operation. For example, a programmable processor can be configured by providing suitable executable code; a dedicated logic circuit can be configured by suitably connecting logic gates and other circuit elements; and so on.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Computer programs incorporating various features of the present invention may be encoded on various computer readable storage media; suitable media include magnetic disk or tape, optical storage media such as compact disk (CD) or DVD (digital versatile disk), flash memory, and the like. Computer readable storage media encoded with the program code may be packaged with a compatible device or provided separately from other devices. In addition, program code may be encoded and transmitted via wired optical, and/or wireless networks conforming to a variety of protocols, including the Internet, thereby allowing distribution, e.g., via Internet download. Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

As described above, one aspect of the present technology is the gathering and use of data available from various sources to improve software application and the software development processes. The present disclosure contemplates that in some instances, this gathered data may include personal information data that uniquely identifies or can be used to contact or locate a specific person. Such personal information data can include demographic data, location-based data, telephone numbers, email addresses, twitter ID's, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used for software energy diagnostics to improve energy consumption of particular software applications. Accordingly, use of such personal information data enables users to improve a particular application used by a user. Further, other uses for personal information data that benefit the user are also contemplated by the present disclosure. For instance, health and fitness data may be used to provide insights into a user's general wellness or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of collecting and processing energy consumption reports, the present technology can be configured to allow users to select to “opt in” or “opt out” of participation in the collection of personal information data during registration for services or anytime thereafter. In another example, users can select not to provide mood-associated data for energy consumption reports. In yet another example, users can select to limit the length of time mood-associated data is maintained or entirely prohibit the development of a baseline mood profile. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, energy consumption reports may be obtained based on non-personal information data or a bare minimum amount of personal information, such as the content being requested by the device associated with a user, other non-personal information available from other sources, or publicly available information.

Although the invention has been described with respect to specific embodiments, it will be appreciated that the invention is intended to cover all modifications and equivalents within the scope of the following claims.

1. A method comprising:
receiving, by a computing device, sensor measurements from each of one or more sensors, wherein the sensor measurements correspond to characteristics of a user during performance of a task;
for each sensor of the one or more sensors:
deriving, from the sensor measurements of the sensor, a set of features predictive of a cognitive load of the user;
generating, from the set of features, a self-attention vector that characterizes each feature of the set of features relative to another feature of the set of features; and
defining a feature vector from the set of features and the self-attention vector;
generating, from the feature vector of at least one sensor of the one or more sensors, an input feature vector;
generating, by a trained machine-learning model using the input feature vector, an indication of the cognitive load of the user during performance of the task; and
outputting, by the computing device, the indication of the cognitive load of the user.

2. The method of claim 1, wherein generating the input feature vector includes deriving a tensor product of the feature vector of the self-attention vector and the set of features.

3. The method of claim 1, wherein generating the input feature vector includes:
aggregating the feature vector of each of the one or more sensors.

4. The method of claim 1, further comprising:
executing a feature projection on the set of features, wherein the feature projection is executed before the self-attention vector is generated.

5. The method of claim 1, wherein deriving the set of features of a first sensor of the one or more sensors includes:
filtering the sensor measurements based on a predetermined frequency relative to a type of the first sensor;
executing an artifact removal process to remove artifacts in the sensor measurements; and
extracting, from the sensor measurements, a plurality of features using a spectral density analysis.

6. The method of claim 1, wherein the computing device is a mobile device and a first sensor of the one or more sensors is positioned within a wearable device.

7. The method of claim 1, further comprising:
normalizing the self-attention vector according to a softmax function before defining the feature vector.

8. A system comprising:
one or more processors
a non-transitory computer-readable medium storing instructions that when executed by the one or more processors, cause the one or more processors to perform operations including:
receiving, by a computing device, sensor measurements from each of one or more sensors, wherein the sensor measurements correspond to characteristics of a user during performance of a task;
for each sensor of the one or more sensors:
deriving, from the sensor measurements of the sensor, a set of features predictive of a cognitive load of the user;
generating, from the set of features, a self-attention vector that characterizes each feature of the set of features relative to another feature of the set of features; and
defining a feature vector from the set of features and the self-attention vector;
generating, from the feature vector of at least one sensor of the one or more sensors, an input feature vector;
generating, by a trained machine-learning model using the input feature vector, an indication of the cognitive load of the user during performance of the task; and
outputting, by the computing device, the indication of the cognitive load of the user.

9. The system of claim 8, wherein generating the input feature vector includes deriving a tensor product of the feature vector of the self-attention vector and the set of features.

10. The system of claim 8, wherein generating the input feature vector includes:
aggregating the feature vector of each of the one or more sensors.

11. The system of claim 8, further comprising:
execute a feature projection on the set of features, wherein the feature projection is executed before the self-attention vector is generated.

12. The system of claim 8, wherein deriving the set of features of a first sensor of the one or more sensors includes:
filtering the sensor measurements based on a predetermined frequency relative to a type of the first sensor;
executing an artifact removal process to remove artifacts in the sensor measurements; and
extracting, from the sensor measurements, a plurality of features using a spectral density analysis.

13. The system of claim 8, wherein the computing device is a mobile device and a first sensor of the one or more sensors is positioned within a wearable device.

14. The system of claim 8, further comprising:
normalizing the self-attention vector according to a softmax function before defining the feature vector.

15. A non-transitory computer-readable medium storing instructions that when executed by a processor, cause the processor to perform operations including:
receiving, by a computing device, sensor measurements from each of one or more sensors, wherein the sensor measurements correspond to characteristics of a user during performance of a task;
for each sensor of the one or more sensors:
deriving, from the sensor measurements of the sensor, a set of features predictive of a cognitive load of the user;
generating, from the set of features, a self-attention vector that characterizes each feature of the set of features relative to another feature of the set of features; and
defining a feature vector from the set of features and the self-attention vector;
generating, from the feature vector of at least one sensor of the one or more sensors, an input feature vector;
generating, by a trained machine-learning model using the input feature vector, an indication of the cognitive load of the user during performance of the task; and
outputting, by the computing device, the indication of the cognitive load of the user.

16. The non-transitory computer-readable medium of claim 15, wherein generating the input feature vector includes deriving a tensor product of the feature vector of the self-attention vector and the set of features.

17. The non-transitory computer-readable medium of claim 15, wherein generating the input feature vector includes:
aggregating the feature vector of each of the one or more sensors.

18. The non-transitory computer-readable medium of claim 15, further comprising:
execute a feature projection on the set of features, wherein the feature projection is executed before the self-attention vector is generated.

19. The non-transitory computer-readable medium of claim 15, wherein deriving the set of features of a first sensor of the one or more sensors includes:
filtering the sensor measurements based on a predetermined frequency relative to a type of the first sensor;
executing an artifact removal process to remove artifacts in the sensor measurements; and
extracting, from the sensor measurements, a plurality of features using a spectral density analysis.

20. The non-transitory computer-readable medium of claim 15, wherein the computing device is a mobile device and a first sensor of the one or more sensors is positioned within a wearable device.
